(19) United States
(12) Patent Application Publication
(10) Pub. No.: US 2014/0368352 A1
(43) Pub. Date: Dec. 18, 2014

(54) METHOD AND SYSTEM FOR AUTOMATED INTERACTIVE GATEWAY SYSTEM

(71) Applicant: Authentidate Holding Corp., Berkeley Heights, NJ (US)

(72) Inventors: O'Connell Julien Benjamin, Marlboro, NJ (US); Carl Richard Duda, New York, NY (US); Winston Hong Lieu, Holmdel, NJ (US)

(21) Appl. No.: 14/302,889

(22) Filed: Jun. 12, 2014

Related U.S. Application Data
(60) Provisional application No. 61/834,306, filed on Jun. 12, 2013.

(51) Int. Cl. A61B 5/00

(52) U.S. Cl. CPC A61B 5/0022; A61B 5/7405
USPC 340/870.02

(57) ABSTRACT

Embodiments of the present teachings disclose method, system, and programs that utilize a gateway and an optional interactive voice response system to allow remote monitoring of a patient's vital signs.

### CROSS REFERENCE TO RELATED APPLICATIONS

The present application claims the benefit of U.S. Provisional Application Ser. No. 61/834,306, filed on 12 Jun. 2013, which is incorporated herein by reference in its entirety.

### BACKGROUND OF THE INVENTION

1. Technical Field

This invention generally relates to monitoring a user at a remote location and more specifically to the use of a combined gateway and interactive voice response system to remotely monitor patients from a remote location.

2. Discussion of Technical Background

Remote patient monitoring allows patients who do not require the immediate services of a hospital or of a primary care physician to remain in their own locations, preferably their homes where they may be monitored remotely, including their vital signs, to ensure ongoing health and compliance with a prescribed treatment option. Many patients may benefit from the use of home monitoring to maintain their prescribed treatments and to monitor their long term treatment plans. Such remote monitoring ensures that a patient's vital signs are being tracked and monitored while at the same time ensuring that their conditions do not worsen or stop responding to prescribed treatment plans.

Remote monitoring, including vital sign monitoring, allows a patient to be tracked in a more timely and cost effective manner than if the patient was required to travel to a healthcare facility. This is important in long term monitoring and treatment situations.

Some systems to monitor patients remotely are known. Systems such as that of U.S. Pat. No. 5,441,047, to David et al. issued Aug. 15, 1995, discloses an ambulatory patient health monitoring system for monitoring a remotely-located healthcare patient from a central station. The system includes instruments at the remote location for measuring the medical condition of the patient. The medical condition may correspond to health parameters, such as heart rate, respiratory rate, pulse oximetry and blood pressure. The system of David however, requires proprietary equipment that includes a first audiovisual camera disposed at the patient location and a second audio-visual camera disposed at the central station. Audio and video information is transmitted between the patient's remote location and the central station via a communications network. The display located at the patient's remote location and at the central station allow a patient and the healthcare worker to observe each other simultaneously and to convey instructions and updates. However, this requires staffing of health care providers for what are often routine measurements and monitoring. The system of David requires face to face communications between the patient and a health care provider to ensure proper monitoring. Accordingly, a need exists for a system that is less expensive, easier to operate and does not require live medical personnel to interface with monitored patients.

Other systems rely on expensive proprietary hardware and provide the user with an interactive terminal display that displays instructions for the patient to monitor the user's specific protocols. These user specific protocols must be configured for each patient and often require frequent changes to the system to ensure compliance. They do not provide a method for reminding the patients and for following up on missing or incomplete data. A need exists for a system that verifies patient compliance, authenticates users and provide patients with reminders and follow up to remain in compliance with the prescribed protocols.

Accordingly, there is a need for systems and methods that can centralize patient protocols, interact with a patient in a easy and inexpensive manner, provide patient authentication and patient reminders and that interacts with various sensors and instruments without the need for direct communication between a health care worker and the patient.

### SUMMARY OF THE INVENTION

The teachings disclosed herein relate to methods, systems, and programming for remotely monitoring patient vital signs utilizing interactive voice response systems, vital sign sensors, a data gateway, and a central portal to manage patient information and disease management plans.

In an embodiment the interactive voice response system utilizes natural voice recordings or synthesized voices to deliver content that may include disease management protocols, reminders, questions and answers to patient inquiries. In another embodiment the interactive voice response system allows for data entry utilizing voice responses or keypad entry. In an embodiment, the system allows for automatic entry of vital sign data via the interactive voice response system or automatic vital sign data entry via the data gateway.

In an embodiment, the portal may detect missing vital sign data from the gateway and generate reminder calls to patients using the interactive voice response system letting them know that they missed a measurement and to schedule follow up calls.

In an embodiment, the interactive voice response system prompts the patient to verify their identity via a user identification interface coupled to the data gateway. In an embodiment, the data gateway supports both wired and wireless communications. In an embodiment, the vital sign data is automatically submitted to the portal and combined with other information obtained via the interactive voice response system. In an embodiment, the time and date of the received vital sign data is compared to a disease management protocol to determine the validity of the data. The compared data may be used to provide feedback to the patient regarding their monitoring regime.

In an embodiment, the data gateway may provide audio and visual alerts to patients as reminders to obtain vital sign data. In an embodiment, the data transmission may be via wired or wireless connections and may be transmitted in an encrypted or unencrypted manner.

In an embodiment, a patient monitoring system for remotely monitoring the patient's vital signs is disclosed. The system comprises a sensor for measuring the patient's vital signs, a data gateway for collecting the patient's vital signs, a patient response system; and a portal for processing the patient's vital signs, wherein the data gateway communicates the collected patient vital signs to the portal in response to a patient management protocol. In an embodiment, the presence, status, maintence record, and other pertinent data pertaining to the sensor are collected by the data gateway to the portal. The sensor status data may be used to provide the patient on follow up or corrective actions required for the sensor.

In an embodiment, the presence, status, maintenance record, and/or other pertinent data relating to the sensor may be collected by the data gateway to the portal. The sensor status data may be used to notify the patient or the device regarding follow up, maintenance or corrective actions required or recommended for the sensor. In an embodiment, a method for remotely monitoring a patient's vital signs is disclosed the method comprises the steps of contacting, via a patient response system, the patient to be monitored, authenticating, via a data gateway, the patients identity, initiating, on a server, a disease management protocol for the patient, obtaining the patient's vital signs, via a sensor connected to the data gateway, in accordance with the disease management protocol, verifying a criteria about the obtained patient's vital signs; and communicating the patient's obtained vital signs to the server. In an embodiment, the sensor is a multipurpose sensor, such as a tablet, laptop, or other smart device.

In an embodiment, the authenticating is accomplished by a patient identification number, a patient biometric identifier, a voice recognition system or an equipment identifier. In an embodiment, the patient response system schedules reminder interactions with the patient to remind the patient to take vitals.

Additional novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 depicts a high level network view of a remote monitoring system, according to an embodiment of the present teaching;

FIG. 2 depicts a block diagram of a gateway, according to an embodiment of the present teaching;

FIG. 3 depicts a block diagram of voice interface unit, according to an embodiment of the present teaching;

FIG. 4 is a flowchart of an exemplary process for patient monitoring, according to an embodiment of the present teaching;

FIG. 5 depicts a high level view of a patient monitoring system with an interactive voice response system and an interactive gateway, according to an embodiment of the present teaching and

FIG. 6 depicts a general computer architecture on which the present teaching can be implemented.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

Systems and methods of the present disclosure may be used to remotely monitor a patients health and vital signs at a remote location. The system and method comprises a series of home based sensors, a gateway to interact with the sensors and to communicate over a network to a server, an interactive voice response system that may communicate with the same or a different server over the same or different network, and a server which may house patient databases, that include patient authentication information, specific patient protocols, and disease management protocols.

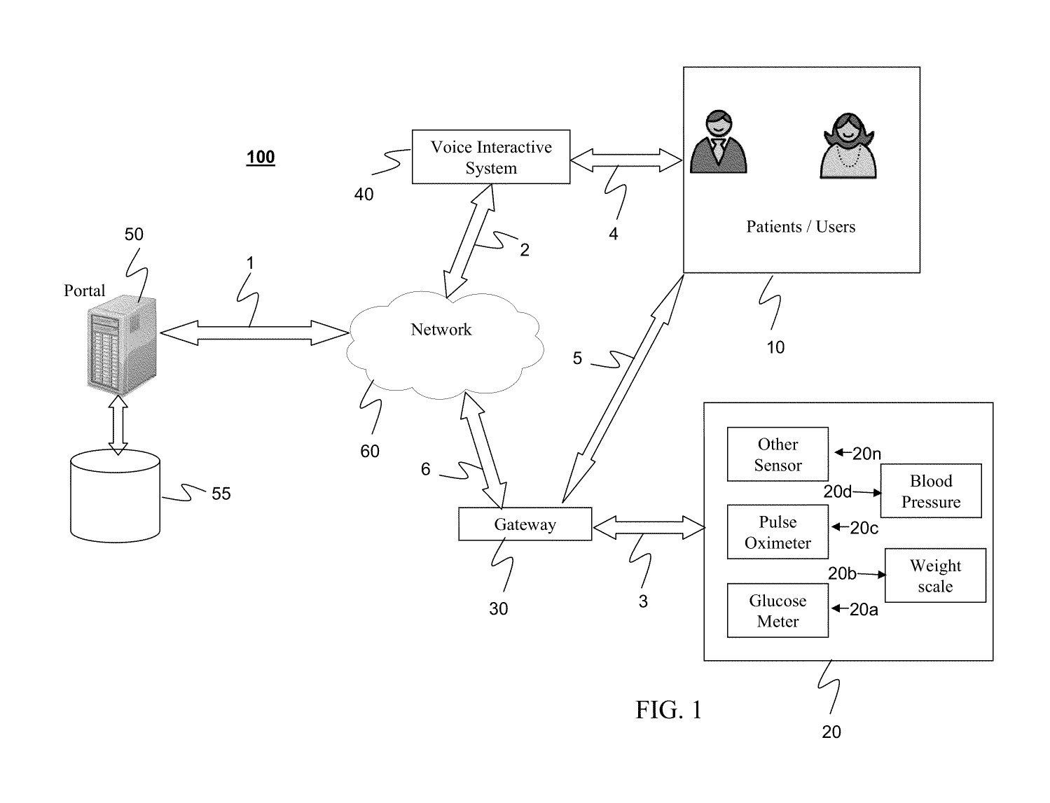

FIG. 1 depicts system 100 which allows users/patients 10 to be remotely monitored by system 100 at their home location. System 100 comprises sensors 20, gateway 30, voice interactive system 40, portal 50 and network 60. Sensors 20 may comprise sensors 20a-20n. Sensor 20a may be a glucose meter, 20b a weight scale, 20c a pulse oximeter, 20d a blood pressure monitor and 20n any other type of sensor such as a thermometer, EKG monitor, auditory monitor, etc. The sensors 20a-20n may be a single monitoring unit or it may be individual monitoring devices. The sensors 20a-20n may all be used by patient 10 every time monitoring is required or may be used sequentially and at different times and frequencies throughout the monitoring periods.

Sensors 20 may be connected to or communicate with gateway 30 via interface 3 by any known means. Interface 3 may be a wired connection or it may be a short range wireless connection such as by radio frequency or infrared. Interface 3 may communicate over WiFi, Bluetooth™ or any other communications protocol. Interface 3 may have a single interface to all the sensors 20a-20n in sensor 20 or may communicate directly with each individual sensor 20a-20n.

Gateway 30 supports both wired and wireless connectivity with various peripherals such as sensors 20 via USB, serial or Bluetooth™. Sensors 20 may be traditional sensors, electronic sensors, or sensors incorporated into a multi-purpose device. Sensor 20 may be a device running a software application to perform the device function. Sensor 20 may be a smartphone, tablet, personal device or other smart device. Gateway 30 communicates to network 60 via wired or wireless connection 6. Additionally and/or alternatively, gateway 30 may communicate wired or wirelessly with portal 50 via connection 1 or 6. Connections 1 and 6 may be via any known communications medium such as modem, cellular, ethernet, WIFI, PSTN or VOIP. Network 60 can be a local area network (LAN), a wide area network (WAN), a public network, a private network, a proprietary network, a Public Telephone Switched Network (PSTN), the Internet, a wireless network, a virtual network, or any combination thereof. A network may also include various network access points, e.g., wired or wireless access points such as base stations or Internet exchange points through which a data source may connect to the network in order to transmit information via the network.

In an embodiment, network 60 is a wireless wide area network, including a network that employs a cellular-based wireless standard, such as CDMA 2000, EV-DO, EV-DV, GSM, GPRS, EDGE, HSPDA, UMTS (Universal Mobile TeleComm. System), LTE (3GPP Long Term Evolution), or UMB (Ultra Mobile Broadband) network access technology. In other embodiments, the network 60 is a LAN (Local Area Network), a WLAN (Wireless Local Area Network) (e.g., Wi-Fi®), or a WiMAX® network.

Gateway 30 works in conjunction with voice interface system 40 as an optional accessory. Gateway 30 may be assigned to a specific patient and may comprise a unique gateway ID that is associated with a specific patient at portal 50 and stored in database 55. Gateway 30 may comprise audible and visual alert reminders in the form of lights, illuminated LEDs, tones, colors, etc. Gateway 30 may also provide secure data transfer via connection 6 to network 60. Additionally and/or alternatively, gateway 30 may comprise an authentication interface for patients 10 to confirm their identity to system 100. Authentication may include, but not be limited to keypad entry, biometric sensors, audio authentication, or any other way to confirm the identify of patient 10.

Voice interactive system 40 may be a interactive voice response system that communicates with patients 10 via a traditional audio device such as a telephone or mobile phone. Voice interactive system 40 may communicate over connection 4 which may be a VOIP system, a PSTN, a POTS line, a cellular network or any other wired or wireless communications link. Voice interactive system 40 may communicate directly with portal 50 or via network 60. Voice interactive system 40 comprises a voice recognition module and a DTMF or touchtone module to interact with and interpret patient 10 responses. Voice interactive system 40 interacts with patient 10's standard telephone unit located in the home and does not require any special user interface unit. A proprietary base unit may be used in place of a telephone base unit.

Voice interactive system 40 allows for patient voice responses or key pad entry. Voice interactive system 40 allows for entry by patient 10 of patient information, such as password, name, status changes, or any other type of requested information. Voice interactive system 40 also allows for manual entry of sensor 20 data if, for example, gateway 30 is not properly transmitting or a sensor is not communicating directly with the gateway.

Voice interactive system 40 may also be used to contact patient 10 directly and provide reminders or to request missing data. For example, in an embodiment, voice interactive system 40 may automatically generate a reminder for patient 10 to take vital signs using sensors 20 during session calls with the patient and it may also schedule follow up reminder calls for the patient 20.

Portal 50 may be a single server or computer or a group of servers or computers dedicated to system 100. Portal 50 may also be part of a network that shares resources. Portal 50 may house patient information including specific disease management protocols for each patient on the system. Additionally, and/or alternatively, portal 50 may store patient personal information, password information, equipment information, scheduling information and any other information related to patents 10. This information may be stored in portal 50 or in a separate database 55. Database 55 may be a single database or may be a series of databases. Database 55 may be relational or flat and may be directly coupled to portal 50 or connected over a network.

In an embodiment, portal 50 will store and associate the data collected for an individual patient 10. It will also manage and maintain a disease management protocol (DMP) for each individual patient 10. The DMP's on portal 50 may relate to patient specific plans or may be related to treatment specific plans. For example, a DMP for a patient may be formed based on the patients specific condition, age, weight, etc, while a treatment plan DMP may be based on a course of treatment for the underlying condition regardless of the specific patient.

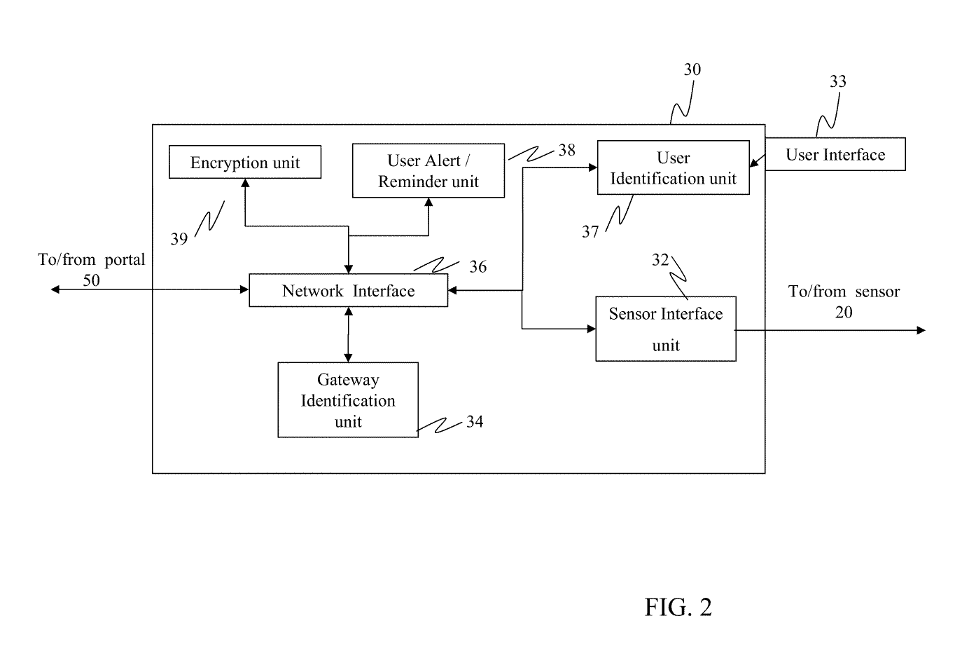

FIG. 2 depicts a block diagram of gateway 30. Gateway 30 comprises sensor interface unit 32, user interface 33, gateway identification unit 34, network interface 36, user interface unit 37, user alert/reminder unit 38 and encryption unit 39. In an embodiment, sensor interface unit 32 interfaces with senor unit 20 and may send and/or receive sensor data from the various sensors 20a-20n. Communications between sensor interface unit 32 and senor 20 may be wired or wireless. The communications between sensor interface unit 32 and sensors 20 may comprise patient vital data gathered by sensors 20a-20n. It may also be software or firmware updates for sensors 20a-20n or inventory data, status data, or self test data or any other type of digital communications between system 100 and the sensor 20.

User identification unit 37 allows the patient 10 to verify their identity to system 100 via gateway 30. User identification unit 37 may interface with a user interface 33 connected directly to gateway 30 by a wired or wireless connection. User interface 33 may be a biometric sensor, such as a thumb or palm print reader, a retina scanner or a voice recognition system, or it may be a keypad type entry device to allow patient 10 to enter an alphanumeric password.

Gateway identification unit 34 is used to identify the specific gateway 30 to portal 50. It may contain a preset serial number or identification number or may have a code generator. Gateway identification unit 34 will respond to portal 50's request via network interface 36 when the information is requested. It may also periodically send information to portal 50 to provide system status.

Network interface 36 monitors and routes the data via network 60 or directly to portal 50 and all data to/from gateway 30 is communicated through network interface 36. User alert/reminder unit 38 may provide user 10 with audio and/or visual alerts to take a specific action. When gateway 30 receives instructions from portal 50, user alert/reminder unit 38 may illuminate an LED or a instructional alert and/or generate an audio signal for patient 10. The alert may be a reminder to patient 10 that it is time to take vital signs or to take medication. It may be an instruction to contact a care provider for a follow up, or any other type alert or simple instruction. In an embodiment, gateway 30 comprises an encryption unit 39 that can encrypt and/or decrypt all messaging being conveyed between portal 50 and gateway 30. Encryption unit 39 may be necessary where personal or medical information is being conveyed over network 60 to ensure patient privacy and compliance with local law. Further, all data communicated to gateway 30 may be encrypted by encryption unit 39 to ensure system security and integrity.

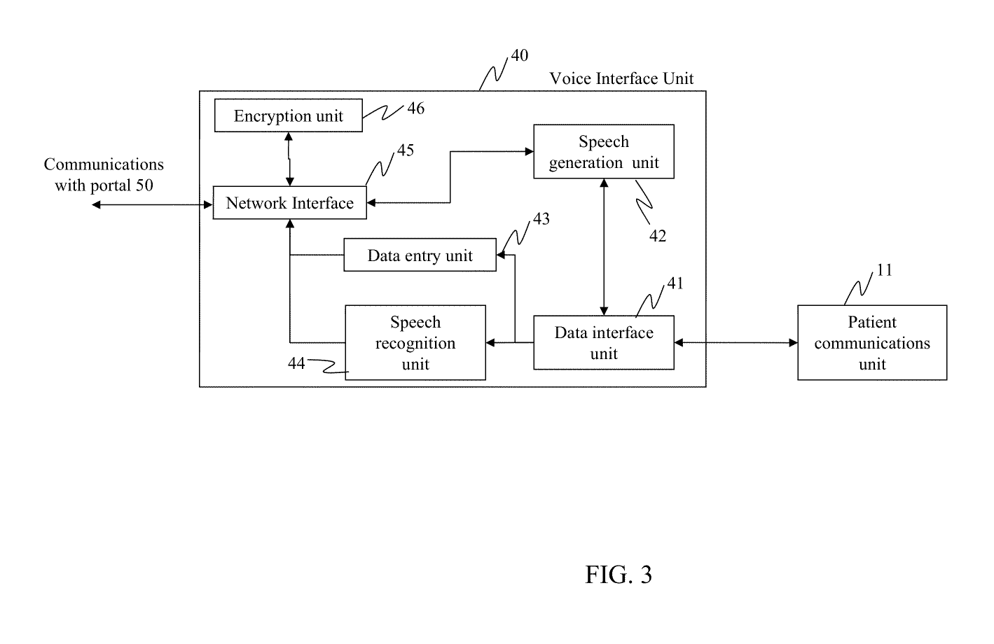

FIG. 3 depicts a block diagram of voice interactive system 40. Voice interactive system 40 comprises data interface 41, speech generation unit 42, data entry unit 43, speech recognition unit 44, network interface 45 and encryption unit 46. Also connected to voice interactive system 40 may be patient communications module 11, which may be a wired or wireless telephone or other voice or keypad interface unit.

Data interface unit 41 allows voice interactive system 40 to communicate with patient communication unit 11 via a standard PSTN, VOIP, or other network connection. Speech generation unit 42 in an embodiment allows the conversion of data to spoken speech for the patient to listen to via patient communication unit 11. based on the patient's specific protocol, portal 50 requires certain information and may provide instructions and or reminders to patient 10. Speech generation unit 42 converts these instructions into a series of verbal commands or requests for data and or information from patient 10. For example, system 100 may be monitoring patient 10's blood pressure and voice interactive system 40 may contact patient 10 twice a day to ask if they have taken their blood pressure. Similarly, voice interactive system 40 may contact patient 10 to remind them to take medications or to follow other medical protocol or to certain follow up actions relating to the sensors 20. In an embodiment, these types of auditory messages may or may not be prerecorded and may be generated by speech generation unit 42 based on a series of instructions.

Data entry unit 43 allows for the interpretation of keypad entries by user 10 via device 11. User 10 may be prompted to enter a number on the telephone keypad and such an entry will be interpreted by data entry unit 43 and conveyed to portal 50. This type of data entry is typically input as Dual Tone Multi-Frequency (DTMF) input or any other similar type input. Speech recognition unit 44 similarly accepts voice responses from patient 10 and interprets them into a response that can be transmitted via network interface 45 to portal 50. Speech recognition unit 44 allows for the interpretation of patient answers in response to the questions presented via speech generation unit 42. Network interface 45 communicates directly or via network 60 with portal 50. This connection may be wired or wireless and may be carried out over one or several local or wide area networks. Encryption unit 46 encrypts patient data transmitted to or from voice interactive system 40 to ensure network integrity and patient security.

Utilizing the voice interactive system 40 and the gateway 30, the portal 50 is able to monitor and gather the necessary data to implement a specific DMP for patient 10. System 100 can send alerts and reminders to patent 10, as outlined by the DMP, that they need to take medical measurements utilizing sensor 20, or that they failed to collect data at a prescribed time. In an embodiment, system 100 may also alert patient 10 that their expected readings are not within the guidelines for their respective DMP and that they should follow up with additional care. Alerts and/or messages may be sent via system 100 or may be sent to additional devices in the form of text messages, e-mail alerts, voice messages or any other means of communication.

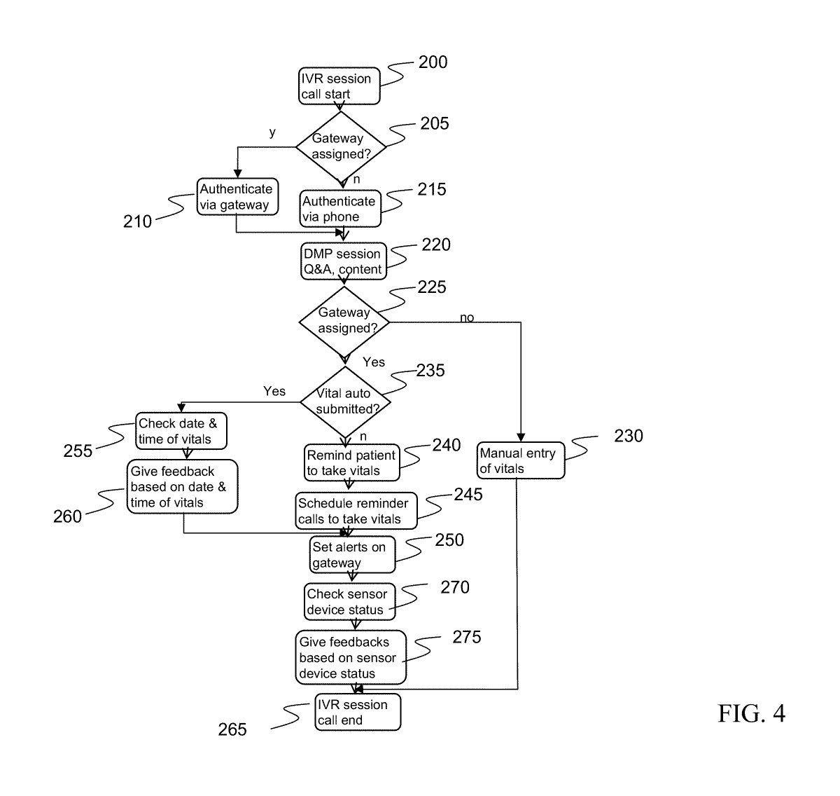

FIG. 4 depicts the steps of an embodiment of system 100. The process depicted begins when portal 50, based on a patient DMP, determines it is time to gather information from patient 10. Portal 50 contacts patient 10 via voice interactive system 40 which places a call at step 200 to user 10 at a predestinated telephone number. Portal 50 at step 205 determines if a gateway 30 is associated with patient 10 and/or with patient 10's predestinated telephone number. If a gateway 30 is present at patient 10's location, gateway identification unit 34 provides gateway authentication to portal 50 to verify the correct gateway and location. Gateway identification unit 34 may provide a unique serial number associated with gateway 30 and/or may respond to a request from portal 50 when interrogated.

Furthermore, in an embodiment, gateway 30 may have a user identification unit 37 that comprises a biometric sensor, a keypad, or any other type of interfaces that allows the user 10 to verify their identification to system 100. Once the user is identified and the gateway authenticated, system 100 may proceed.

If at step 205, it is determined form the information in database 550 that there is no gateway assigned to patient 10, at step 215 authentication may be provided via voice interactive system 40. Authentication may come in the form of a spoken password that is interpreted by speech recognition module 42 or by patient 10 entering an alphanumeric password via a telephone keypad which is interpreted by data entry module 44. This information may be verified at voice interactive system 40 or may be conveyed to portal 50 for verification.

Once authentication is completed, at step 220, a DMP session for patient 10 is started by portal 50. The DMP session may be seeking specific sensor measurements or may be addressing a series of questions and/or instructions from or for patient 10. If the DMP is seeking senor measurement data and a gateway was assigned, then if vital sensor data was previously collected, the vital sensor data is submitted to the portal at step 235. The date and time of the previously stored sensor data will be checked at step 255 and at step 260 feedback may be provided based on the date and time of the stored vital data. For example, the sensor data may be too old to rely on or it may have been recorded to early or to late in the day. Similarly, reminders may be given to patient 10 of the course of treatment defined in the patient's DMP. If all the data is available, the voice interactive system will end the session at step 265.

If it is determined at step 235 that there is no stored vital data, then at step 240 a reminder in the form of an audio message may be conveyed to patient 10 with follow up instructions. At step 245, portal 50 will schedule reminder calls and at step 250 alerts will be set up to alert the patient via the gateway 30 to follow up on collecting vital signs. Gateway 30 reminders may be audio tones or visual indicators. These messages and instructions may also be given to patient 10 by portal 50 using scheduled reminder calls.

At step 270, the status data of the sensors 20 may be collected by gateway 30 and sent to portal 50. Based on the status data, in step 275, portal 50 may provide messages or instructions to patient 10 regarding any necessary or recommended follow up or corrective actions required for sensors 20. These messages and instructions may also be provided to patient 10 by portal 50 using scheduled reminder calls.

If no gateway was assigned at step 225, the voice interactive system 40 may allow for manual entry of vital information at step 230. The vital information may be conveyed via keypad entries form user 10's telephone to voice interactive system 40 or may be conveyed orally utilizing the speech recognition module of voice interactive system 40. Once all vital information is gathered, then the interactive voice session will be terminated by voice interactive system 40.

In operation gateway 30 may alert and/or prompt patient 10 that they need to provide authentication or that they need to take a measurement using one of the sensors 20a-20n. Additionally and/or alternatively, utilizing voice interactive system 40 in conjunction with gateway 30, system 100 can monitor patients 10 in a cost effective and efficient manner.

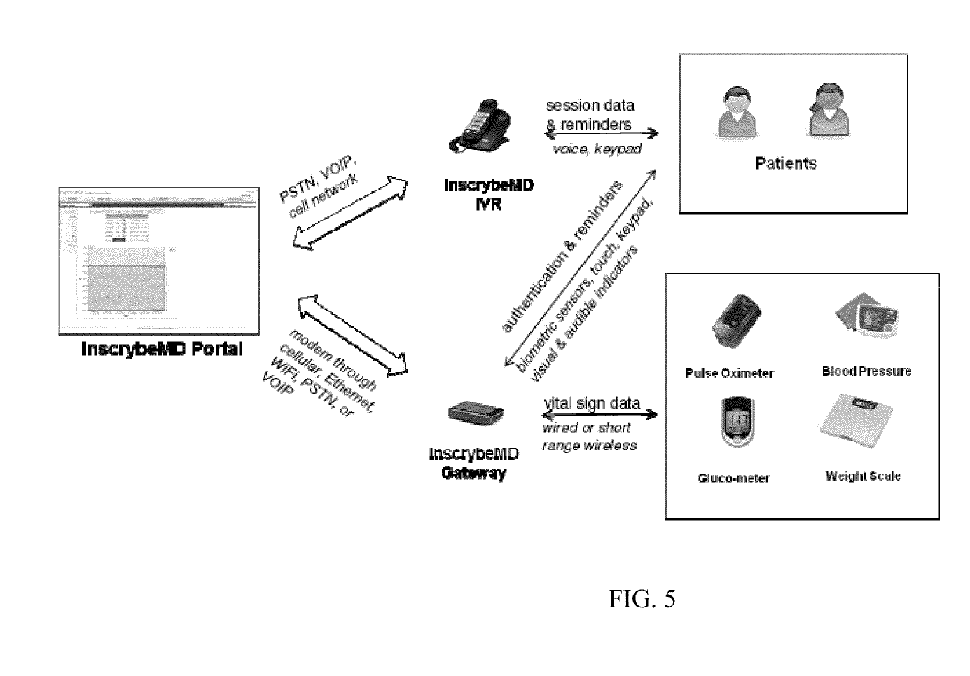

FIG. 5 depicts an alternative embodiment of system 100.

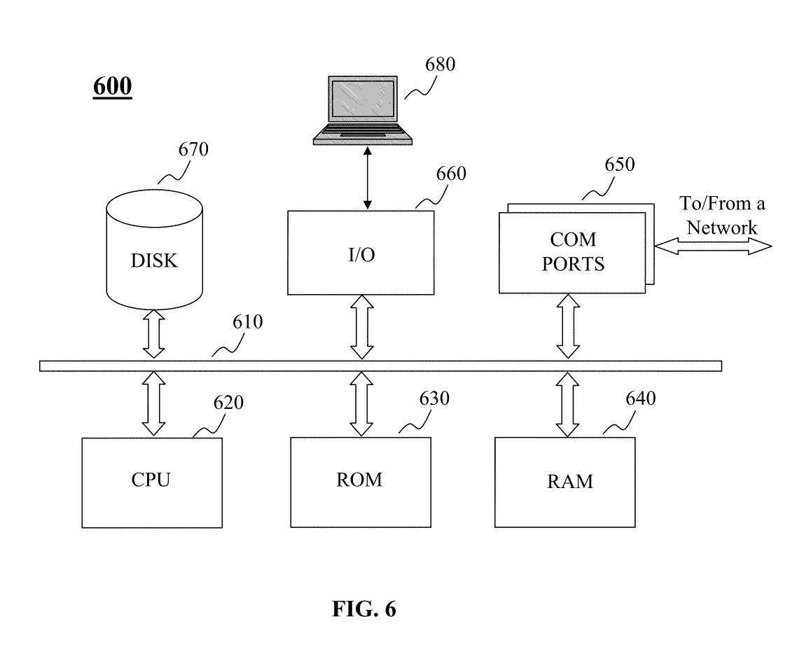

FIG. 6 depicts a general computer architecture on which the present teaching can be implemented and has a functional block diagram illustration of a computer hardware platform that includes user interface elements. The computer may be a general-purpose computer or a special purpose computer. This computer 600 can be used to implement any components of patient monitoring architecture as described herein. Different components of the system in the present teaching can all be implemented on one or more computers such as computer 600, via its hardware, software program, firmware, or a combination thereof. Although only one such computer is shown, for convenience, the computer functions relating to remote patient monitoring may be implemented in a distributed fashion on a number of similar platforms, to distribute the processing load.

The computer 600, for example, includes COM ports 602 connected to and from a network connected thereto to facilitate data communications. The computer 600 also includes a central processing unit (CPU) 604, in the form of one or more processors, for executing program instructions. The exemplary computer platform includes an internal communication bus 606, program storage and data storage of different forms, e.g., disk 1208, read only memory (ROM) 1210, or random access memory (RAM) 612, for various data files to be processed and/or communicated by the computer, as well as possibly program instructions to be executed by the CPU. The computer 600 also includes an I/O component 614, supporting input/output flows between the computer and other components therein such as user interface elements 616. The computer 600 may also receive programming and data via network communications.

Hence, aspects of the method of remote patient monitoring, as outlined above, may be embodied in programming. Program aspects of the technology may be thought of as “products” or “articles of manufacture” typically in the form of executable code and/or associated data that is carried on or embodied in a type of machine readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to tangible “storage” media, terms such as computer or machine “readable medium” refer to any medium that participates in providing instructions to a processor for execution.

Hence, a machine readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Those skilled in the art will recognize that the present teachings are amenable to a variety of modifications and/or enhancements. For example, although the implementation of various components described above may be embodied in a hardware device, it can also be implemented as a software only solution. In addition, the components of the system as disclosed herein can be implemented as a firmware, firmware/software combination, firmware/hardware combination, or a hardware/firmware/software combination.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings

1. A patient monitoring system for remotely monitoring a patient's vital signs comprising:
a sensor for measuring the patient's vital signs;
a data gateway for collecting the patient's vital signs;
a patient response system; and
a portal for processing the patient's vital signs,
wherein the data gateway communicates the collected patient vital signs to the portal in response to a patient management protocol.

2. A method for remotely monitoring a patient's vital signs comprising:
contacting, via a patient response system, the patient to be monitored;
authenticating, via a data gateway, the patients identity;
initiating, on a server, a disease management protocol for the patient;
obtaining the patient's vital signs, via a sensor connected to the data gateway, in accordance with the disease management protocol;
verifying a criteria about the obtained patient's vital signs; and
communicating the patient's obtained vital signs to the server.

3. The system of claim 1, wherein the patient response system is an active voice response system.

4. The method of claim 2, wherein the patient response system is an active voice response system.

5. The method of claim 2, further comprising:
detecting the absence of vital sign data in the sensor;
contacting the patient through the patient response system; and
reminding the patient to submit vital sign data using the sensors.

6. The method of claim 2, further comprising:
providing, via the patient response system, instructions and feedbacks to the patient based on a date and a time of the vital sign data.

7. The method of claim 2, further comprising:
providing, via the patient response system, instructions and feedbacks to the patient based on status data of the sensor received from the data gateway.

8. The patient monitoring system of claim 1, wherein the sensor is a multipurpose sensor.

9. The patient monitoring system of claim 1, wherein the sensor is an application on a smart device.

10. The method of claim 2, wherein the authenticating is accomplished by at least one of the following:
a patient identification number, a patient biometric identifier, a voice recognition system and an equipment identifier.

11. The method of claim 2, wherein the patient response system schedules reminder interactions with the patient to remind the patient to take vitals.
